(19) United States
(12) Patent Application Publication
(10) Pub. No.: US 2007/0197955 A1
(43) Pub. Date: Aug. 23, 2007

(54) MUCOUS MEMBRANE ADHESION-TYPE IONTOPHORESIS DEVICE

(75) Inventors: Hidero Akiyama, Shibuya-Ku (JP); Mizuo Nakayama, Shibuya-Ku (JP); Takehiko Matsumura, Shibuya-Ku (JP); Akihiko Matsumura, Shibuya-Ku (JP)

Correspondence Address: SEED INTELLECTUAL PROPERTY LAW GROUP PLLC, 701 FIFTH AVE, SUITE 5400, SEATTLE, WA, 98104, US

(73) Assignee: TRANSCUTANEOUS TECHNOLOGIES INC., Shibuya-ku (JP)

(21) Appl. No.: 11/549,031

(22) Filed: Oct. 12, 2006

Related U.S. Application Data
(60) Provisional application No. 60/730,234, filed on Oct. 24, 2005.

(30) Foreign Application Priority Data
Oct. 12, 2005 (JP) 2005-297745

(51) Int. Cl. A61N 1/30

(52) U.S. Cl. 604/20

(57) ABSTRACT

An iontophoresis device may include: an electric power source; a first electrode assembly coupled to the electric power source; and a second electrode assembly as a counter electrode of the first electrode assembly. The first electrode assembly may include an adhesive portion that facilitates contact between a mucous membrane and the first electrode assembly. The adhesive portion may be placed on a portion of an end surface portion of an electrode assembly, and may be made from a material that exhibits adhesiveness after absorbing an aqueous medium.

### CROSS REFERENCE TO RELATED APPLICATION

The present application claims the benefit under 35 U.S.C. § 119(e) of U.S. Provisional Patent Application Ser. No. 60/730,234, entitled “MUCUS MEMBRANE ATTACHABLE IONTOPHORESIS DEVICE,” filed Oct. 24, 2005, assigned to the same assignee as the present application, and which is incorporated herein by reference in its entirety.

### TECHNICAL FIELD

The present disclosure generally relates to the field of iontophoresis, and in particular but not exclusively, to an iontophoresis device capable of administering an ionic active agent into a mucous membrane that contains moisture.

### BACKGROUND INFORMATION

A method of delivering an ionic active agent placed at a predetermined location on a biological interface, such as the skin or a mucous membrane, of a subject and into the body by employing an electromotive force sufficient to drive the ionic active agent is known as iontophoresis. JP 63-35266 A, for example, describes an iontophoresis device.

Positively charged ions may be driven (transported) into the biological interface on the side of an anode (positive electrode) of an iontophoresis device. On the other hand, negatively charged ions may be driven into the biological interface on the side of a cathode (negative electrode).

Several types of iontophoresis devices have been proposed to date (such as in: JP 63-35266 A; JP 04-297277 A; JP 2000-229128 A; JP 2000-229129 A; JP 2000-237327 A; JP 2000-237328 A; and WO 03/037425 A1, for example).

To provide stable, efficient active agent delivery, an iontophoresis device may need to be fixed in place on a biological interface. When the biological interface is a mucous membrane that contains moisture, however, it may be difficult to securely fix the iontophoresis device in place, which may reduce any therapeutic affect available.

### BRIEF SUMMARY

In one aspect, the present disclosure is directed to an iontophoresis device that may be capable of efficient, stable administration of an ionic active agent to a mucous membrane that contains surface moisture. The iontophoresis device of one embodiment may comprise: an electric power source; a first electrode assembly coupled to the electric power source; and a second electrode assembly as a counter electrode of the first electrode assembly. The first electrode assembly may include an adhesive portion on at least a portion of an end surface of the first electrode assembly. The adhesive portion may comprise a member or material that exhibits adhesive characteristics upon absorption of an aqueous medium, and may help to facilitate close contact between a mucous membrane and the first electrode assembly.

In at least one embodiment, the second electrode assembly may surround an outer peripheral portion of the first electrode assembly. The second electrode assembly may include an adhesive portion on an outer peripheral portion thereof.

In another aspect, the present disclosure is directed to a method of operating an iontophoresis device, comprising: placing the first electrode assembly and the second electrode assembly on a mucous membrane; energizing the iontophoresis device with the electric power source; and allowing the first electrode assembly to release the ionic active agent.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a view showing the outline of one embodiment of an iontophoresis device.

FIG. 2 is a perspective view showing the rear surface side of one embodiment of an electrode assembly.

FIG. 3 is a view showing the outline of one embodiment of an iontophoresis device.

FIG. 4 is a perspective view showing the rear surface side of one embodiment of an iontophoresis device.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with iontophoresis devices, controllers, voltage or current sources and/or membranes have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment,” or “an embodiment,” or “another embodiment” means that a particular referent feature, structure, or characteristic described in connection with the embodiment is included in at least one embodiment. Thus, the appearances of the phrases “in one embodiment,” or “in an embodiment,” or “another embodiment” in various places throughout this specification are not necessarily all referring to the same embodiment. Furthermore, the particular features, structures, or characteristics may be combined in any suitable manner in one or more embodiments.

It should be noted that, as used in this specification and the appended claims, the singular forms “a,” “an,” and “the” include plural referents unless the content clearly dictates otherwise. Thus, for example, reference to a system for evaluating an iontophoretic active agent delivery including “a controller” includes a single controller, or two or more controllers. It should also be noted that the term “or” is generally employed in its sense including “and/or” unless the content clearly dictates otherwise.

As used herein the term “membrane” means a boundary, a layer, barrier, or material, which may, or may not be permeable. The term “membrane” may further refer to an interface. Unless specified otherwise, membranes may take the form a solid, liquid, or gel, and may or may not have a distinct lattice, non cross-linked structure, or cross-linked structure.

As used herein the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein the term “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane substantially permits the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, an anion exchange membrane substantially permits the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein, the term “bipolar membrane” means a membrane that is selective to two different charges or polarities. Unless specified otherwise, a bipolar membrane may take the form of a unitary membrane structure, a multiple membrane structure, or a laminate. The unitary membrane structure may include a first portion including cation ion exchange materials or groups and a second portion opposed to the first portion, including anion ion exchange materials or groups. The multiple membrane structure (e.g., two film structure) may include a cation exchange membrane laminated or otherwise coupled to an anion exchange membrane. The cation and anion exchange membranes initially start as distinct structures, and may or may not retain their distinctiveness in the structure of the resulting bipolar membrane.

As used herein, the term “semi-permeable membrane” means a membrane that is substantially selective based on a size or molecular weight of the ion. Thus, a semi-permeable membrane substantially passes ions of a first molecular weight or size, while substantially blocking passage of ions of a second molecular weight or size, greater than the first molecular weight or size. In some embodiments, a semi-permeable membrane may permit the passage of some molecules a first rate, and some other molecules a second rate different than the first. In yet further embodiments, the “semi-permeable membrane” may take the form of a selectively permeable membrane allowing only certain selective molecules to pass through it.

As used herein, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “gel matrix” means a type of reservoir, which takes the form of a three dimensional network, a colloidal suspension of a liquid in a solid, a semi-solid, a cross-linked gel, a non cross-linked gel, a jelly-like state, and the like. In some embodiments, the gel matrix may result from a three dimensional network of entangled macromolecules (e.g., cylindrical micelles). In some embodiment a gel matrix may include hydrogels, organogels, and the like. Hydrogels refer to three-dimensional network of, for example, cross-linked hydrophilic polymers in the form of a gel and substantially composed of moisture. Hydrogels may have a net positive or negative charge, or may be neutral.

A used herein, the term “reservoir” means any form of mechanism to retain an element, compound, pharmaceutical composition, active agent, and the like, in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force and/or current into the biological interface. A reservoir may also retain an electrolyte solution.

A used herein, the term “active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a active agent, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent. In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, ionizable and/or neutral therapeutic active agent and/or pharmaceutical acceptable salts thereof. In yet other embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. While other active agents may be polarized or polarizable, that is exhibiting a polarity at one portion relative to another portion. For instance, an active agent having an amino group can typically take the form an ammonium salt in solid state and dissociates into a free ammonium ion (NH4+) in an aqueous medium of appropriate pH. The term “active agent” may also refer to neutral agents, molecules, or compounds capable of being delivered via electro-osmotic flow. The neutral agents are typically carried by the flow of, for example, a solvent during electrophoresis. Selection of the suitable active agents is therefore within the knowledge of one skilled in the art. Non-limiting examples of such active agents include lidocaine, articaine, and others of the -caine class; morphine, hydromorphone, fentanyl, oxycodone, hydrocodone, buprenorphine, methadone, and similar opiod agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine and ziprasidone as well as other neuroleptica; diabetes drugs such as exenatide; as well as peptides and proteins for treatment of obesity and other maladies.

As used herein and in the claims, the term “subject” generally refers to any host, animal, vertebrate, or invertebrate, and includes fish, mammals, amphibians, reptiles, birds, and particularly humans.

The headings provided herein are for convenience only and do not interpret the scope or meaning of the embodiments.

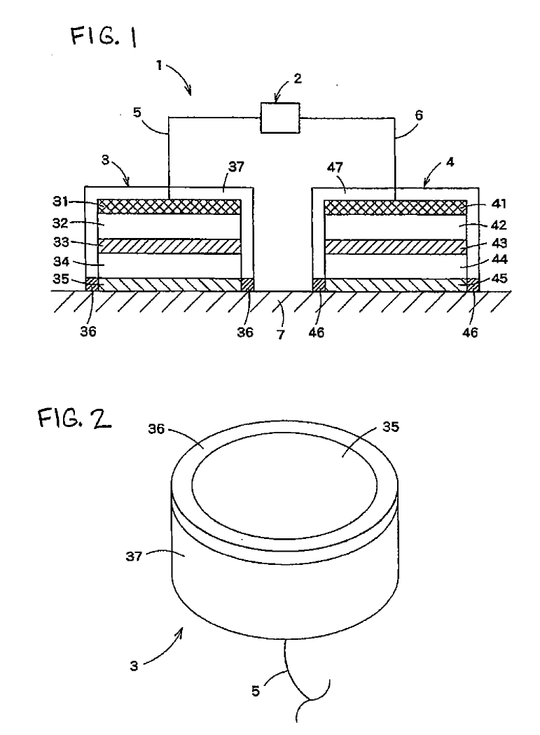

FIG. 1 shows an iontophoresis device 1 arranged on a mucous membrane 7 that contains moisture on and/or in a surface thereof. The iontophoresis device 1 comprises: an electric power source 2; a first electrode assembly 3 coupled to the electric power source 2; and a second electrode assembly 4 as a counter electrode to the first electrode assembly. The first electrode assembly 3 comprises: an electrode 31 coupled via a conductive member 5 to a side of the electric power source 2 having the same polarity as that of an active agent; an electrolyte reservoir 32 that holds an electrolyte solution, the electrolyte reservoir 32 being arranged adjacent to the electrode 31; an ion exchange membrane 33 that selectively passes ions having a polarity opposite that of the ionic active agent, the ion exchange membrane 33 being arranged adjacent to the electrolyte reservoir 32; an active agent reservoir 34 holding the ionic active agent by being impregnated with the ionic active agent, the active agent reservoir 34 being arranged adjacent to the ion exchange membrane 33; an ion exchange membrane 35 that selectively passes ions having the same polarity as that of the ionic active agent, the ion exchange membrane 35 being arranged adjacent to the active agent reservoir 34; and an adhesive portion 36 that facilitates close contact between the mucous membrane 7 and the first electrode assembly 3. The adhesive portion 36 is arranged on at least a portion of an end surface portion. A cover or container 37 made from a material such as a resin film or plastic may be used to house the elements described above.

The second electrode assembly 4 comprises an electrode 41 that acts as a counter electrode to the electrode 31. The electrode 41 is coupled to the electric power source 2 via a conductive member 6. The second electrode assembly 4 also comprises: an electrolyte reservoir 42 that holds an electrolyte solution, the electrolyte reservoir 42 being arranged adjacent to the electrode 41; an ion exchange membrane 43 that selectively passes ions having the same polarity as the ionic active agent, the ion exchange membrane 43 being arranged adjacent to the electrolyte reservoir 42; an electrolyte reservoir 44 that holds an electrolyte solution, the electrolyte reservoir 44 being arranged adjacent to the ion exchange membrane 43; an ion exchange membrane 45 that selectively passes ions having a polarity opposite that of the ionic active agent, the ion exchange membrane 45 being arranged adjacent to the electrolyte reservoir 44; and an adhesive portion 46 that facilitates close contact between the mucous membrane 7 and the second electrode assembly 4, the adhesive portion 46 being arranged on at least a part of an end surface portion. A cover or container 47 made from a material such as a resin film or plastic may be used to house the elements described above.

The adhesive portions 36 and 46 may each comprise a member that exhibits adhesiveness after absorption of an aqueous medium such as water moisture. A release liner (not shown) may be placed on the outer surface of the adhesive portions, and removed before using the iontophoresis device 1.

Alternatively, the second electrode assembly may comprise: an electrode that acts as a counter electrode to the electrode 31; an electrolyte reservoir that holds an electrolyte solution, the electrolyte reservoir being arranged adjacent to the electrode; an ion exchange membrane that selectively passes ions having a polarity opposite that of the ionic active agent, the ion exchange membrane being arranged adjacent to the electrolyte reservoir; and an adhesive portion that facilitates close contact between the mucous membrane 7 and the second electrode assembly 4, the adhesive portion being arranged on at least a part of an end surface portion.

FIG. 2 is a perspective view showing a rear surface side of the first electrode assembly 3. The adhesive portion 36 is arranged on the outer peripheral portion of the ion exchange membrane 35 on the end surface portion of the first electrode assembly 3. It may be advantageous to place the adhesive portion 46 on the outer peripheral portion of the ion exchange membrane 45, on the end surface portion of the second electrode assembly 4. However, each adhesive portion may be disposed in other suitable locations provided that delivery of an ionic active agent is not inhibited. In the embodiment shown in FIG. 1, both electrode assemblies have an adhesive portion. In some embodiments, however, an iontophoresis device 1 may be constructed in such a manner that only the first electrode assembly has an adhesive portion.

Referring to FIG. 1, the first electrode assembly 3 and the second electrode assembly 4 may be arranged on the mucous membrane 7, and energization may be achieved through use of the electric power source 2, thus allowing an ionic active agent to be administered from the first electrode assembly 3 into a subject through the mucous membrane 7. The ionic active agent may be efficiently and stably released because the first electrode assembly 3 is in close contact with the mucous membrane via the adhesive portion.

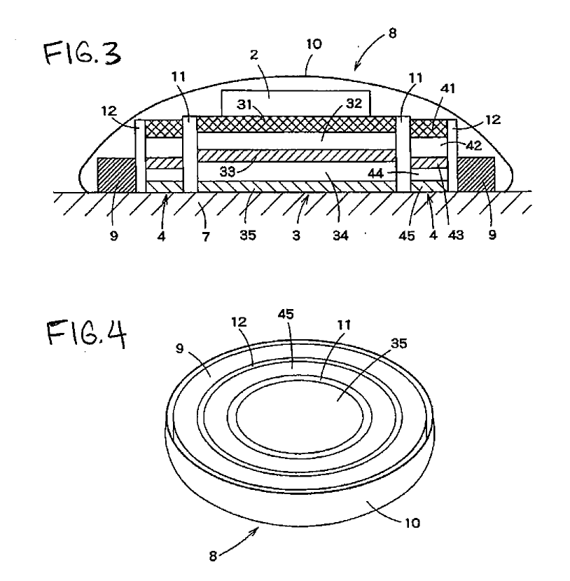

FIG. 3 shows an iontophoresis device 8 arranged on the mucous membrane 7 that contains moisture on and/or in a surface thereof. The iontophoresis device 8 comprises: the electric power source 2; the first electrode assembly 3 coupled to the electric power source 2; the second electrode assembly 4 as a counter electrode of the first electrode assembly 3; and an adhesive portion 9 for bringing the mucous membrane 7 and the iontophoresis device 8 into close contact with each other. A cover or container 10 made from a moisture resistant material such as a resin film or a plastic may be used to house the elements described above. The second electrode assembly 4 may surround the outer peripheral portion of the first electrode assembly 3, and the adhesive portion 9 may surround the outer peripheral portion of the second electrode assembly 4. The iontophoresis device 8 further comprises: a first insulating portion 11 arranged between the first electrode assembly 3 and the second electrode assembly 4; and a second insulating portion 12 arranged between the second electrode assembly 4 and the adhesive portion 9.

The adhesive portion 9 may comprise a member or material that exhibits adhesive characteristics upon absorption of an aqueous medium. A release liner (not shown) may be placed on the outer surface of the adhesive portions, and removed before using the iontophoresis device.

The first electrode assembly 3 and the second electrode assembly 4 that surrounds the outer peripheral portion of the assembly 3 may each be substantially similar to the configurations shown in FIG. 1.

Alternatively, the second electrode assembly may comprise: an electrode that acts as a counter electrode to the electrode 31; an electrolyte reservoir that holds an electrolyte solution, the electrolyte reservoir being arranged adjacent to the electrode; and an ion exchange membrane that selectively passes ions having a polarity opposite that of the ionic active agent, the ion exchange membrane being arranged adjacent to the electrolyte reservoir.

FIG. 4 is a perspective view showing the rear surface side of the iontophoresis device 8. The ion exchange membrane 45 in the second electrode assembly 4 surrounds the outer peripheral portion of the ion exchange membrane 35 of the first electrode assembly 3, and the adhesive portion 9 surrounds the outer peripheral portion of the ion exchange membrane 45 in the second electrode assembly 4. The first insulating portion 11 is arranged between the ion exchange membrane 35 and the ion exchange membrane 45 so that the first electrode assembly 3 and the second electrode assembly 4 are separated from each other. In addition, the second insulating portion 12 is arranged between the ion exchange membrane 45 and the adhesive portion 9 so that the second electrode assembly 4 and the adhesive portion 9 are separated from each other.

The electric power source 2, the first electrode assembly 3, and the second electrode assembly 4 shown in FIG. 4 may be configured integrally, thus making it easier to arrange the iontophoresis device 8 on a mucous membrane. This may be advantageous when the iontophoresis device 8 is used in a small space such as an oral cavity.

In general, the first electrode assembly comprises an active electrode assembly for releasing an ionic active agent, and the second electrode assembly comprises a counter electrode assembly. In some embodiments, however, both electrode assemblies may be constituted to release an ionic active agent.

The adhesive portion of one embodiment may comprise at least one material selected from the group consisting of alginic acid, pectin, lower methoxyl pectin, guar gum, gum arabic, cargeenan, methylcellulose, carboxymethylcellulose sodium, xanthan gum, hydroxypropylcellulose, hydroxypropylmethylcellulose, and crystalline cellulose carmellose sodium, and is more preferably in one embodiment at least one selected from the group consisting of carboxymethylcellulose sodium, xanthan gum, hydroxypropylmethylcellulose, and crystalline cellulose carmellose sodium.

The first insulating portion and the second insulating portion may each comprise a known insulating material such as polystyrene or rubber. WO 03/037425 A1,the contents of which are incorporated herein by reference, describes components of an iontophoresis device in detail.

The above description of illustrated embodiments, including what is described in the Abstract, is not intended to be exhaustive or to limit the embodiments to the precise forms disclosed. Although specific embodiments and examples are described herein for illustrative purposes, various equivalent modifications can be made without departing from the spirit and scope of the disclosure, as will be recognized by those skilled in the relevant art. The teachings provided herein of the various embodiments can be applied to other problem-solving systems devices, and methods, not necessarily the exemplary problem-solving systems devices, and methods generally described above.

For instance, the foregoing detailed description has set forth various embodiments of the systems, devices, and/or methods via the use of block diagrams, schematics, and examples. Insofar as such block diagrams, schematics, and examples contain one or more functions and/or operations, it will be understood by those skilled in the art that each function and/or operation within such block diagrams, schematics, or examples can be implemented in one embodiment, individually and/or collectively, by a wide range of hardware, software, firmware, or virtually any combination thereof. In one embodiment, the present subject matter (such as the power source 2 and related circuitry, for example) may be implemented via Application Specific Integrated Circuits (ASICs). However, those skilled in the art will recognize that the embodiments disclosed herein, in whole or in part, can be equivalently implemented in standard integrated circuits, as one or more computer programs running on one or more computers (e.g., as one or more programs running on one or more computer systems), as one or more programs running on one or more controllers (e.g., microcontrollers) as one or more programs running on one or more processors (e.g., microprocessors), as firmware, or as virtually any combination thereof, and that designing the circuitry and/or writing the code for the software and or firmware would be well within the skill of one of ordinary skill in the art in light of this disclosure.

In addition, those skilled in the art will appreciate that the mechanisms of taught herein are capable of being distributed as a program product in a variety of forms, and that an illustrative embodiment applies equally regardless of the particular type of signal bearing media used to actually carry out the distribution. Examples of signal bearing media include, but are not limited to, the following: recordable type media such as floppy disks, hard disk drives, CD ROMs, digital tape, and computer memory; and transmission type media such as digital and analog communication links using TDM or IP based communication links (e.g., packet links).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese Patent Application Serial No. H03-86002, filed Mar. 27, 1991, having Japanese Publication No. H04-297277, issued on Mar. 3, 2000 as Japanese Patent No. 3040517; Japanese Patent Application Serial No.11-033076, filed Feb. 10, 1999, having Japanese Publication No. 2000-229128; Japanese Patent Application Serial No.11-033765, filed Feb. 12, 1999, having Japanese Publication No. 2000-229129; Japanese Patent Application Serial No. 1-041415, filed Feb. 19, 1999, having Japanese Publication No. 2000-237326; Japanese Patent Application Serial No. 11-041416, filed Feb. 19, 1999, having Japanese Publication No. 2000-237327; Japanese Patent Application Serial No. 11-042752, filed Feb. 22, 1999, having Japanese Publication No. 2000-237328; Japanese Patent Application Serial No. 11-042753, filed Feb. 22, 1999, having Japanese Publication No. 2000-237329; Japanese Patent Application Serial No. 11-099008, filed Apr. 6,1999, having Japanese Publication No. 2000-288098; Japanese Patent Application Serial No. 11-099009, filed Apr. 6,1999, having Japanese Publication No. 2000-288097; PCT Patent Application WO 2002JP4696, filed May 15, 2002, having PCT Publication No WO03037425; U.S. patent application Ser. No. 10/488,970, filed Mar. 9, 2004; Japanese Patent Application 2004/317317, filed Oct. 29, 2004; U.S. Provisional Patent Application Ser. No. 60/627,952, filed Nov. 16, 2004; Japanese Patent Application Ser. No. 2004-347814, filed Nov. 30, 2004; Japanese Patent Application Ser. No. 2004-357313, filed Dec. 9, 2004; Japanese Patent Application Ser. No. 2005-027748, filed Feb. 3, 2005; Japanese Patent Application Ser. No. 2005-081220, filed Mar. 22, 2005; and U.S. Provisional Patent Application Ser. No. 60/754,953, filed Dec. 28, 2005.

Aspects of the embodiments can be modified, if necessary, to employ systems, circuits, and concepts of the various patents, applications, and publications to provide yet further embodiments.

These and other changes can be made to the embodiments in light of the above-detailed description. In general, in the following claims, the terms used should not be construed to limit the invention to the specific embodiments disclosed in the specification and the claims, but should be construed to include all possible embodiments along with the full scope of equivalents to which such claims are entitled. Accordingly, the scope of the invention shall only be construed and defined by the scope of the appended claims.

What is claimed is:

1. An iontophoresis device, comprising:
an electric power source;
a first electrode assembly coupled to the electric power source, the first electrode assembly including:
an adhesive portion having a member that exhibits adhesiveness after absorbing moisture, wherein the adhesive portion is placed on at least a portion of an end surface of the first electrode assembly and wherein the adhesive portion facilitates contact between the first electrode assembly and a mucous membrane of a subject; and
a second electrode assembly as a counter electrode of the first electrode assembly.

2. The iontophoresis device according to claim 1 wherein the first electrode assembly further includes:
an electrode coupled to a side of the electric power source having the same polarity as that of an ionic active agent;
an electrolyte reservoir that holds an electrolyte solution, the electrolyte reservoir placed adjacent to the electrode;
a first ion exchange membrane that selectively passes ions having a polarity opposite that of the ionic active agent, the first ion exchange membrane placed adjacent to the electrolyte reservoir;
an active agent reservoir that holds the ionic active agent, the active agent reservoir placed adjacent to the ion exchange membrane; and
a second ion exchange membrane that selectively passes ions having the same polarity as that of the ionic active agent, the second ion exchange membrane placed adjacent to the active agent reservoir.

3. The iontophoresis device according to claim 2 wherein the adhesive portion is placed on an outer peripheral portion of the ion exchange membrane that selectively passes ions having the same polarity as that of the ionic active agent.

4. The iontophoresis device according to claim 1 wherein the second electrode assembly includes an adhesive portion that facilitates contact between the second electrode assembly and the mucous membrane, wherein the adhesive portion is placed on at least a part of an end surface portion of the second electrode assembly, and wherein the adhesive portion includes a member that exhibits adhesiveness after absorbing an aqueous medium.

5. The iontophoresis device according to claim 4 wherein the second electrode assembly further includes:
an electrode coupled to a side of the electric power source having a polarity opposite that of an ionic active agent;
an electrolyte reservoir that holds an electrolyte solution, the electrolyte reservoir placed adjacent to the electrode; and
an ion exchange membrane that selects ions having a polarity opposite that of the ionic active agent, the ion exchange membrane placed adjacent to the electrolyte reservoir.

6. The iontophoresis device according to claim 4 wherein the second electrode assembly further includes:
an electrode coupled to a side of the electric power source having a polarity opposite that of an ionic active agent;
a first electrolyte reservoir that holds an electrolyte solution, the first electrolyte reservoir placed adjacent to the electrode;
a first ion exchange membrane that selects ions having the same polarity as that of the ionic active agent, the first ion exchange membrane placed adjacent to the electrolyte reservoir;
a second electrolyte reservoir that holds an electrolyte solution, the second electrolyte reservoir being placed adjacent to the ion exchange membrane; and
a second ion exchange membrane that selects ions having a polarity opposite that of the charged ion of the ionic active agent, the second ion exchange membrane being placed adjacent to the electrolyte reservoir.

7. The iontophoresis device according to claim 5 wherein the adhesive portion is placed on an outer peripheral portion of the ion exchange membrane.

8. The iontophoresis device according to claim 6 wherein the adhesive portion is placed on an outer peripheral portion of the second ion exchange membrane.

9. An iontophoresis device, comprising:
an electric power source;
a first electrode assembly coupled to the electric power source; and
a second electrode assembly as a counter electrode of the first electrode assembly, the second electrode assembly surrounding an outer peripheral portion of the first electrode assembly and including:
an adhesive portion having a member that exhibits adhesiveness after absorbing moisture, wherein the adhesive portion surrounds an outer peripheral portion of the second electrode assembly.

10. The iontophoresis device according to claim 9 wherein the first electrode assembly further includes:
an electrode coupled to a side of the electric power source having the same polarity as that of an ionic active agent;
an electrolyte reservoir that holds an electrolyte solution, the electrolyte reservoir placed adjacent to the electrode;
a first ion exchange membrane that selectively passes ions having a polarity opposite that of the ionic active agent, the first ion exchange membrane placed adjacent to the electrolyte reservoir;
an active agent reservoir that holds the ionic active agent, the active agent reservoir placed adjacent to the ion exchange membrane; and
a second ion exchange membrane that selectively passes ions having the same polarity as that of the ionic active agent, the second ion exchange membrane placed adjacent to the active agent reservoir.

11. The iontophoresis device according to claim 9 wherein the second electrode assembly further includes:
an electrode coupled to a side of the electric power source having a polarity opposite that of an ionic active agent;
an electrolyte reservoir that holds an electrolyte solution, the electrolyte reservoir placed adjacent to the electrode; and
an ion exchange membrane that selects ions having a polarity opposite that of the ionic active agent, the ion exchange membrane placed adjacent to the electrolyte reservoir.

12. The iontophoresis device according to claim 9 wherein the second electrode assembly further includes:
an electrode coupled to a side of the electric power source having a polarity opposite that of an ionic active agent;
a first electrolyte reservoir that holds an electrolyte solution, the first electrolyte reservoir placed adjacent to the electrode;
a first ion exchange membrane that selects ions having the same polarity as that of the ionic active agent, the first ion exchange membrane placed adjacent to the electrolyte reservoir;
a second electrolyte reservoir that holds an electrolyte solution, the second electrolyte reservoir being placed adjacent to the ion exchange membrane; and
a second ion exchange membrane that selects ions having a polarity opposite that of the charged ion of the ionic active agent, the second ion exchange membrane being placed adjacent to the electrolyte reservoir.

13. The iontophoresis device according to claim 9, further comprising a first insulating portion placed between the first electrode assembly and the second electrode assembly.

14. The iontophoresis device according to claim 9, further comprising a second insulating portion placed between the second electrode assembly and the adhesive portion.

15. The iontophoresis device according to claim 4 wherein the member exhibiting adhesiveness owing to absorption of the aqueous medium comprises at least one material selected from the group consisting of alginic acid, pectin, low-methoxyl pectin, guar gum, gum arabic, carageenan, methylcellulose, sodium carboxymethylcellulose, xanthan gum, hydroxypropylcellulose, hydroxypropylmethylcellulose, and crystalline cellulose carmellose sodium.

16. The iontophoresis device according to claim 9 wherein the member exhibiting adhesiveness comprises at least one material selected from the group consisting of carboxymethylcellulose, xanthan gum, hydroxypropylmethylcellulose, and crystalline cellulose carmellose sodium.

17. The iontophoresis device according to claim 1 wherein the mucous membrane comprises an oral mucous membrane.

18. A method of operating an iontophoresis device that includes (a) an electric power source, (b) a first electrode assembly coupled to the electric power source and including an adhesive portion having a member that exhibits adhesiveness after absorbing moisture, wherein the adhesive portion is placed on at least a portion of an end surface of the first electrode assembly and wherein the adhesive portion facilitates contact between the first electrode assembly and a mucous membrane of a subject, and (c) a second electrode assembly as a counter electrode of the first electrode assembly, the method comprising:
placing the first electrode assembly and the second electrode assembly on the mucous membrane;
energizing the iontophoresis device using the electric power source; and
allowing the first electrode assembly to release an ionic active agent.
